(19) United States
(12) Patent Application Publication
(10) Pub. No.: US 2009/0180967 A1
(43) Pub. Date: Jul. 16, 2009

(54) ULTRSONICALLY ACTIVE MICROPARTICLES AND METHOD OF USE

(76) Inventors: Eugene Tu, San Diego, CA (US); Donald E. Ackley, Cardiff, CA (US)

Correspondence Address: ROBERT A. PARSONS, 4000 N. CENTRAL AVENUE, SUITE 1220, PHOENIX, AZ, 85012, US

(21) Appl. No.: 12/354,596

(22) Filed: Jan. 15, 2009

Related U.S. Application Data
(60) Provisional application No. 61/021,207, filed on Jan. 15, 2008.

(51) Int. Cl. A61K 49/22

(52) U.S. Cl. 424/9.51; 424/9.5; 424/9.52

(57) ABSTRACT

An ultrasonically active microparticle is taught that includes a porous interior particle having hydrophobic pores, a gas in the hydrophobic pores and a hydrophilic exterior shell surrounding the interior particle forming an ultrasonically active microparticle and allowing the microparticle to be suspended in an aqueous solution.

### FIELD OF THE INVENTION

This invention relates to microparticles.

More particularly, the present invention relates to ultrasonically active microparticles.

### BACKGROUND OF THE INVENTION

Ultrasound examination is a non-invasive technique for observing the inner workings of a living organism such as a human or animal body. While effectively used as a diagnostic procedure for many years, ultrasound images can be difficult to decipher, with many tissues and fluids indistinguishable from adjacent tissues and fluids. To clarify the images produced by ultrasonic examination, contrast agents are used. Currently, gas filled lipid or protein microbubbles (i.e. Definity and Optison) have been developed for use as contrast agents. These gas filled microbubbles as ultrasound contrast agents suffer from poor circulating lifetimes in the body.

### SUMMARY OF THE INVENTION

Briefly, to achieve the desired objects and advantages of the instant invention in accordance with a preferred embodiment thereof, provided is an ultrasonically active microparticle comprising a porous interior particle having hydrophobic pores, a gas in the hydrophobic pores, and a hydrophilic exterior shell surrounding the interior particle, forming an ultrasonically active microparticle and allowing the microparticle to be suspended in an aqueous solution. In a specific aspect, the ultrasonically active microparticle has a size in the range of 0.1 to 20 microns.

In yet another aspect, the ultrasonically active microparticle includes a delivery structure containing a substance, the delivery structure is carried by the ultrasonically active microparticle. The delivery structure includes at least one nanopacket containing the substance, the nanopacket attached to the outer shell of the ultrasonically active microparticle. The delivery structure can include a delivery shell enclosing the microparticle and the substance.

In another aspect, a method of using ultrasonically active microparticles is provided. The method comprises the steps of providing a plurality of ultrasonically active microparticle in an aqueous solution. Each ultrasonically active microparticle includes a porous interior particle having hydrophobic pores, a gas in the hydrophobic pores, and a hydrophilic exterior shell surrounding the interior particle, forming an ultrasonically active microparticle and allowing the microparticle to be suspended in the aqueous solution. The solution of ultrasonically active microparticles is then introduced to a desired site. In a specific aspect, the solution is introduced intravenously, and the solution is allowed to circulate in the circulatory system to reach the desired site. Ultrasonic imaging and manipulation of the ultrasonically active microparticles can be used to facilitate introduction to the desired site and indications of arrival at the site.

### BRIEF DESCRIPTION OF THE DRAWINGS

Specific objects and advantages of the invention will become readily apparent to those skilled in the art from the following detailed description of a preferred embodiment thereof, taken in conjunction with the drawings in which:

FIG. 1 is a simplified sectional schematic of an ultrasonically active microparticle according to the present invention;

FIG. 2 is a simplified sectional schematic of a delivery structure containing an ultrasonically active microparticle;

FIG. 3 is a simplified sectional schematic of another delivery structure;

FIG. 4 is a schematic diagram illustrating neutralization of an ink particle according to the present invention;

FIG. 5 is a schematic diagram illustrating another embodiment of the neutralization of an ink particle;

FIG. 6 is a schematic diagram illustrating another embodiment of the neutralization of an ink particle;

FIG. 7 is a schematic diagram illustrating another embodiment of the neutralization of an ink particle;

FIG. 8 is a schematic diagram illustrating another embodiment of the neutralization of an ink particle;

FIG. 9 is a schematic diagram illustrating another embodiment of the neutralization of an ink particle; and

FIG. 10 is a schematic diagram illustrating another embodiment of the neutralization of an ink particle.

### DETAILED DESCRIPTION OF A PREFERRED EMBODIMENT

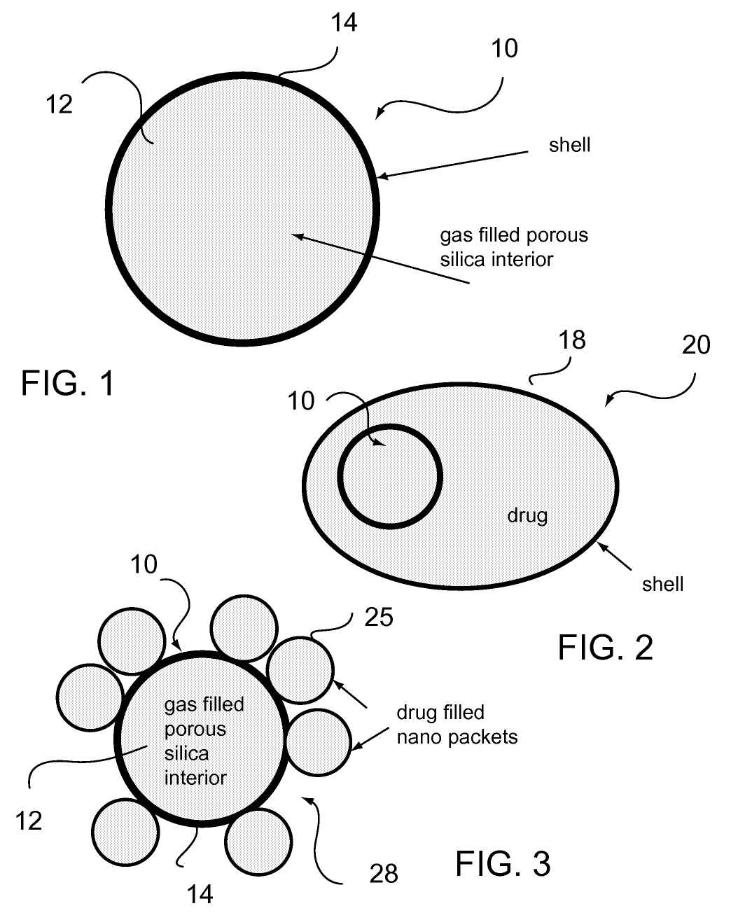

Turning now to the drawings in which like reference characters indicate corresponding elements throughout the several views, attention is directed to FIG. 1 which illustrates an ultrasonically active microparticle generally designated 10. Ultrasonically active microparticle 10 includes a gas filled porous interior particle 12 surrounded by an exterior shell 14.

Interior particle 12 is a highly porous material which is filled with a gas such as air, an inert gas, a perfluorocarbon, or the like. In order to suppress capillary action and retain porosity, highly hydrophobic materials such as fluorosilane are incorporated into the pores to prevent filling in aqueous solution. The silanes also serve to present a hydrophobic surface to the outside environment. Particles 12 are coated with a hydrophilic polymer such as polyvinylpyrrolidone to form shell 14, which allows microparticles 10 to be suspended in aqueous solution. To be effective in imaging as well as therapeutic applications, as will be discussed presently, ultrasonically active microparticle 10 is preferably of a size in the range of 0.1 to 20 microns, and more preferably in the range of 1 to 10 microns.

Microparticles 10 are composed of organic and/or inorganic monomers/polymers reactants, such as tetraethylorthosilicate, alginate, chitosan, and organosiloxanes. Gas filled particles 12 are preferably created by a simple sol-gel process using 1 or 2 step hydrolysis and condensation reactions. Porogens or other templating molecules may be included to alter porosity, pore size, and particle morphology. For example porous interior particle 12 can have a porous shell around a larger interior pore or hollow interior, or be generally uniformly porous throughout, as desired. Aerogels are created by extracting the solvent at temperatures and pressures greater than the solvent critical temperature where capillary surface tension is negligible so drying doesn't result in matrix collapse and crosslinking. Another method that uses ambient temperature/pressure conditions involves reaction of free silanols with hydrophobic silanes such as trimethylchlorosilane or tridecafluorotriethoxysilane which prevents irreversible collapse of the gel matrix. After drying at 80 C, the matrix springs back to an aerogel. Aerogels with interpenetrating pore networks (sponge-like) or porous shells (porous hollow balls) can be formed by varying reaction conditions and templating molecules. If necessary, the templating molecules may be removed from the aerogel by baking at high temperature (˜600 C) to remove organic molecules.

While silica sols are the preferred material, organic molecules, such as CTAB, pluronic, etc. can also be incorporated to vary the pore size or create a hollow interior. It has also been found that highly porous PLGA [(poly(lactic-co-glycolic acid)], an FDA approved medical polymer, can be made and employed as interior particle 12.

A silane crosslinker containing an ester moiety, for example, can be added during the sol-gel process to create a aerogel matrix that is stable when filled with dry gas but will hydrolyze and accelerate degradation when filled with aqueous fluids. PLGA naturally hydrolyzes in aqueous fluids so matrix modifications are unnecessary

These interior particles 12 must be subsequently treated with hydrophobic materials such as silanes in order to create a highly hydrophobic interior pore structure and surface. Without silanizing particles 12, aqueous fluids will displace the gasses in the porous structure rendering them useless as a contrast agent.

The bulk sol-gel matrix formed can be milled and sieved to produce porous particles 12 of the desired size and monodispersity. Sol-gel particles may be formed on a droplet generator before the condensation phase, which potentially leads to a more efficient synthesis process and a more monodisperse distribution of particle sizes and shapes. Using various templating materials such as CTAB, pluronic, etc., hollow round spherical microparticles may be produced.

Gases of interest, such as air, inert gases, or perfluorocarbons, are exchanged into porous particle 12 under pressure or vacuum and retained there when the microparticles are introduced to aqueous solution through the action of the highly hydrophobic pores. In order to allow dispersion of the hydrophobic particles 12 into aqueous solutions, and to potentially enhance the ultrasonic activity of the particles, outer shell 14 of a suitable hydrophilic polymer such as polyvinylpyrrolidone, polyvinyl alcohol, polysaccharide, acrylamide, thiophene and the like is added. It has also been found that outer shell 14, also defined as a coating, of Bovine Serum Albumin (0.5-2 mg/ml BSA) can be used. Streptavidin was also tested and found to adhere to interior particle 12. A lipid membrane can also be used for this purpose.

If the microparticles are to be suspended in organic solutions then the aerogel matrix can be silanized with hydrophobic moieties on the surface.

Ultrasonically active microparticles 10 scatter ultrasound because of the large change in sound velocity between the aqueous solution and the gas filled interior of the particle. Sound induced pressure changes cause the gas to expand and contract, leading to strong resonances with the applied sound wave. In a manner that is quite different from the gas microbubbles currently in use, which expand and contract like a balloon, shell 14 of microparticle 10 can only expand, as its minimum interior dimension is defined by porous particle 12. This creates an asymmetric resonance, creating a rich harmonic spectrum and enhancing the ultrasonic activity of microparticle 10, especially in ultrasound systems where excitation occurs at one frequency and scattered signal is detected at a harmonic frequency. Thus, ultrasonically active microparticle 10 is highly visible during therapeutic uses as will be described presently.

At sufficiently high sound powers, the hydrophobic surface forces that keep the aqueous solution out are overcome and particles 12 fill, losing their ultrasonic activity. At high applied sound powers, the process is catastrophic, leading to destructive gas microjets and shattering microparticles 10. Alternatively, the microjets can penetrate the shells at high velocity. These processes can be used to liberate carried materials, such as drugs or the process can be used to destroy blood clots, tumors and the like. Thus, the ultrasonically active microparticles 10 can be injected or otherwise supplied to a desired location and ultrasonically manipulated.

Ultrasonically active microparticles 10 can be manipulated in ultrasonic fields to enhance their efficacy in imaging and therapeutics. For example, because they are stable compared to existing lipid or protein encapsulated gas bubbles, they can be moved and steered using an externally applied ultrasound field. In addition, when an ultrasound field is applied, ultrasonically active microparticles 10 tend to spin and tumble. These effects can be particularly useful in therapeutic applications where microparticles 10 can impact, for example target tumors.

Thus, there are two major advantages to microparticles 10. First, in contrast to existing microbubbles, microparticles 10 are extremely stable in addition to being ultrasonically active. Second, this stability enables them to be incorporated into secondary, drug containing structures for combined imaging (contrast agent) and ultrasonically activated drug delivery.

In addition, microparticles 10 can be targeted to specific cells by binding targeting moieties such as antibodies or antibody fragments, oligonucleotides, peptides and the like to the porous surface of microparticle 10 or to the outer shell 14.

By producing the sol-gels from precursors that incorporate magnetically active materials such as iron or gadolinium, particles 12 can be produced which act as contrast agent both for ultrasound and MRI. Similarly, by incorporating fluorescent dyes into the sol-gel matrix or outer shell 14, ultrasonically active microparticles 10 that are both ultrasonically and optically active may be created. By adding these additional active modalities, combined therapeutic effects may be achieved. For example, MRI has been used to heat metal microparticles up to release drug and/or to destroy tumors.

Turning now to FIG. 2, in a further embodiment, microparticles 10 are nested within a delivery shell 18 which contains a substance of interest such as a drug, forming a delivery structure 20. In order to combine a contrast agent with a drug payload in a delivery structure 20, ultrasonically active microparticles 10 can be nested within a second, aqueous filled delivery shell 18. Structure 20 can be manufactured using droplet generator technology. Microparticles 10 are suspended in a drug containing aqueous solution which is pumped down a center channel of a droplet generator. An immiscible or semi-miscible fluid containing polymers, proteins, saccharides, lipids or a combination thereof is pumped down side channels to form droplets which encapsulate ultrasonically active microparticles 10 along with the drug in the nested configuration of structure 20. These droplets may be finished off so as to add surface functionalization, different outer layer compositions, or PEG to improve their characteristics within the body. Alternatively, combining an aqueous solution containing ultrasonically active microparticles 10 with a miscible solvent containing lipids, vortexing or sonicating the mixture, and allowing unilamellar vesicles (liposomes) to surround the ultrasonically active microparticles 10, forms the nested structures of structure 20. Vesicle size is determined by the lipid composition, temperature, and reaction time. Other well-known methodologies for forming giant liposomes, such as solvent evaporation, freeze-thaw, or rehydration may also be applied to surround the ultrasonically active micropoarticles. In yet another alternative, fully formed liposomes in a mixture of solvent and water may be introduced into the solution containing ultrasonically active microparticles, with the mixture being subsequently sonicated or vortexed at elevated temperatures (approximately 50 C), resulting in a lipid membrane surrounding the ultrasonically active microparticle.

Referring now to FIG. 3, as an alternative to nesting ultrasonically active microparticles 10 in structure 20, preformed nanopackets 25 containing a substance of choice such as a drug may be attached to outer shell 14 of ultrasonically active microparticles 10, forming delivery structure 28. Nanopackets 25 can be held in place using hydrophobic interactions, electrostatic attraction, or self assembled using surface functionalization of ultrasonically active microparticles 10 outer shell. Electrostatic attraction can be enhanced by creating oppositely charged ultrasonically active microparticles 10 and nanopacket 25 surfaces, for example by choosing appropriate lipid or polymer compositions. Various surface functionalization moieties can include simple reactive groups (i.e. amine or maleimide) that are compatible with the aqueous polymer shells and can form a covalent bond with the nanopackets 25. Homo- or bi-functional crosslinkers can be used to minimize steric interactions and introduce a cleavable bond (i.e disulfide or nucleic acid). Affinity components such as biotin/streptavidin, or oligonucleotides for selective hybridization can be used to self assemble the delivery structure 28. Antibody-antigen reactions could potentially be used to attach materials as well. For electrostatic attachment of drug containing liposomes, PVP coated microparticles were suspended in 0.01× PBS, pH 9 to deprotonate the ring and add a negative charge. The drugs containing liposomes contained cationic lipids to add a positive charge and create the electrostatic binding force. In this specific example, the lipid membranes contained 25% DOTAP, a positively charged cationic lipid. The solubility of the drugs contained inside nanopackets 25 does not have an impact on the design of particles 12.

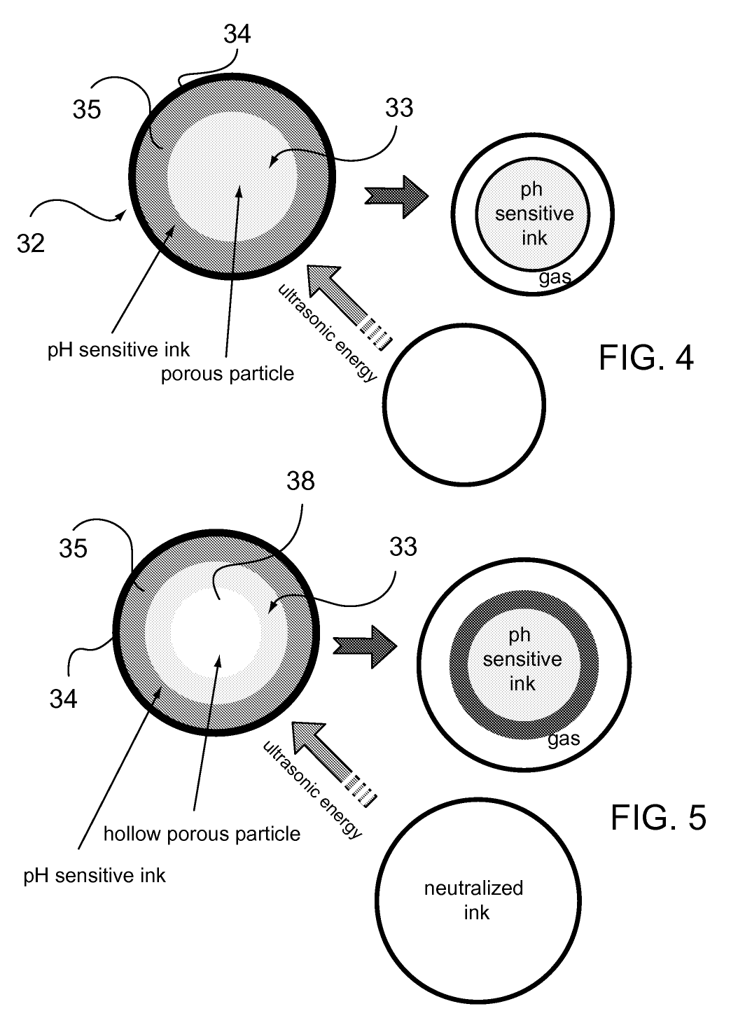

An example of a specific application of a delivery structure 20 can be employed in the tattooing industry. Specifically, delivery structure 20 includes dyes or inks as the substance of choice to be delivered. In this case, the delivery is postponed until removal of the tattoo is desired. As will be described presently, the delivery structure 20 is positioned as in the conventional tattooing process. If the tattoo becomes undesirable, ultrasonically active microparticle 10 within delivery structure 20 is ultrasonically activated, delivering the ink which is subsequently neutralized. Thus, with reference to FIG. 4, delivery structure 20 includes ultrasonically active microparticles 10 contained within delivery shell 18 encapsulating ink 30. By exciting ultrasonically active microparticles 10 with ultrasonic energy at a frequency that resonates therewith, ultrasonically active microparticles 10 can be fragmented, penetrating delivery shell 18 and allowing ink to leak out. Alternatively, ultrasonically active microparticles 10 include gas containing particle 12 which when excited, the gas is driven from the porous material as high velocity jets, again penetrating the delivery shell 18 and releasing the ink. In another embodiment, ultrasonic energy overcomes a hydrophobic barrier on the outer surface of outer shell 14, allowing it to fill within. By properly functionalizing the porous surface of ultrasonically active microparticles 10 to modify the the pH of the ink, pH sensitive inks and dyes may be made transparent in the interior of ultrasonically active microparticles 10. Other mechanisms to render dyes transparent include oxidation/reduction and formation/breaking of chemical bonds. The ink pigment could be removed by environmental changes (pH), chemical modification (degradation), or dispersal (lymphatic drainage).

In an alternative embodiment, ink 30 could be replaced in delivery structure 20 by a drug, embedded into the skin, and delivered sub-dermally using ultrasonic excitation, thereby minimizing the need for multiple injections of drug in some therapies, or for cosmetic applications.

In yet another alternative embodiment, ink 30 could be replaced in delivery structure 20 by an anticancer drug, a solution of delivery structure 20 intraveneously administered, the delivery structures allowed to accumulate in the vicinity of a cancerous tumor, and the anticancer drug released by ultrasound so as to attack the tumor with minimal side effects to the patient. The anticancer drugs could include small molecule drugs such as 5-fluorouracil or doxorubicin, or genetic material such as siRNA or antisense sequences. Target tumors include glioblastomas, and tumors attacking the liver, pancreas, or bladder.

Referring now to FIG. 4, a delivery structure 32 including a porous silica ultrasonically active microparticle 33 made by a sol-gel process or some other means, is illustrated. The internal surface area is structured or functionalized to create an environment that will be acidic (or basic) when filled with water. Each ultrasonically active microparticle 33 is filled with a gas that has poor solubility in water. Ultrasonically active microparticle 33 is silanized to make its surface and pores hydrophobic and is nested inside a hollow polymer or lipid (sugar, carbohydrate, protein) delivery shell 34 which also encapsulates a pH sensitive ink, dye or pigment 35. As a tattoo, structures 32 can be colored in a wide range of colors and are applied to the skin using conventional tattoo machines. When the patient decides to remove the tattoo, the pigmented area is excited using an ultrasound transducer that produces energy at a frequency resonant with the encapsulated ultrasonically active microparticles 33. The ultrasonic energy overcomes the hydrophobic barrier, and ultrasonically active microparticle 33 fills with pigment or ink 35 surrounding it, displacing the gas to the outside. As ultrasonically active microparticle 33 fills, the pH of the ink solution is lowered, rendering the dye or pigment transparent and removing the tattoo. As ink 35 remains encapsulated, there is reduced danger of adverse health effects to the body.

Turning now to FIG. 5, ultrasonically active microparticle 33 includes a centrally located volume 38 and contains additional gas to enhance the ultrasonic resonance. The ink is rendered acidic by material that is washed from the pores of ultrasonically active microparticle 33 and hence neutralized. Alternatively, soluble gases such as CO2 are known to reduced the pH of aqueous solutions. Thus, if ultrasonically active microparticle 33 is filled with such a gas, when the gas is released and solubilized, the pH of the ink will be reduced and the ink will be rendered transparent.

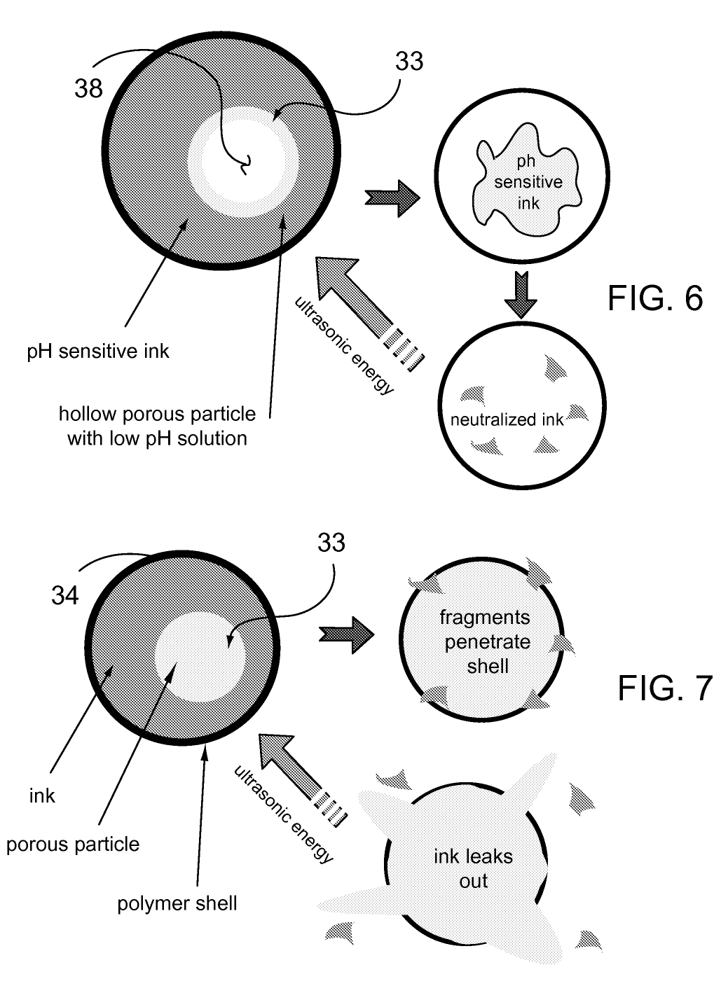

Referring to FIG. 6, in yet another embodiment, centrally located volume 38 of ultrasonically active microparticle 33 is filled with acidic solution, which is released upon excitation by ultrasonic energy. This release may be achieved by the collapse of the hydrophobic barrier, or alternatively by shattering with the applied ultrasound.

Turning to FIG. 7, ultrasonically active microparticle 33 is shattered by the ultrasonic energy. Its fragments penetrate delivery shell 34, allowing the ink to leak out and be neutralized and/or dispersed by the body, in much the same way as laser based systems allow ink to be fragmented and dispersed by the body. In FIGS. 7-10 the dye is released to the body and could be rendered transparent at physiological pH and/or dispersed by the lymphatic system.

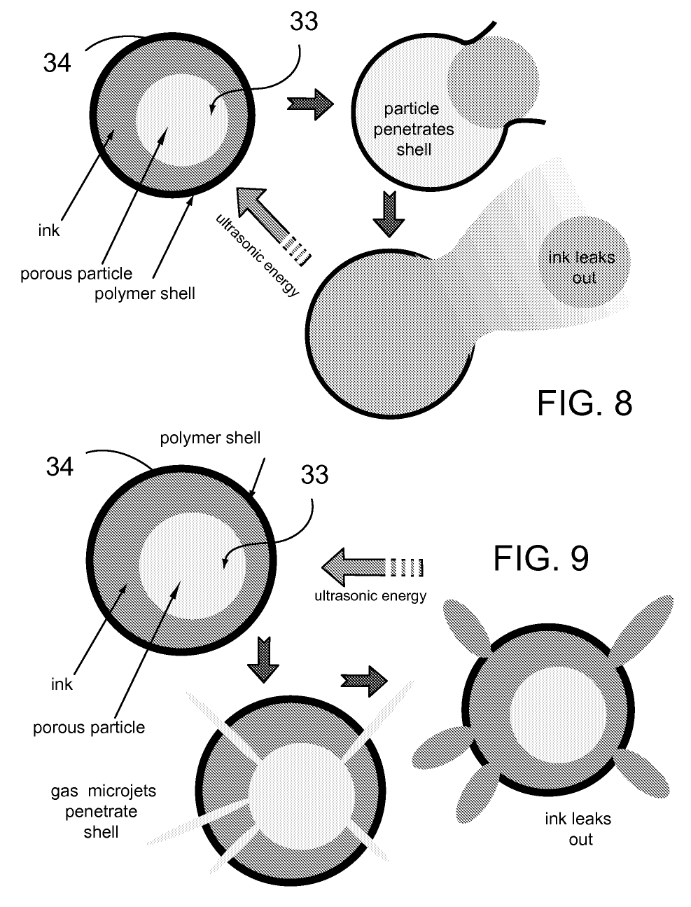

In FIG. 8, upon ultrasonic excitation, ultrasonically active microparticles 33 spin and rotate. Taking advantage of this property, the particle is used to essentially “drill” through delivery shell 34 under ultrasound excitation, efficiently releasing the ink for neutralization by the body.

With reference to FIG. 9, ultrasonically active microparticle 33 can be caused to collapse with ultrasound, with the result that the gas is released in the form shock waves and high energy microjets. By using this property and allowing the microjets to penetrate delivery shell 34, the encapsulated ink may be induced to leak out.

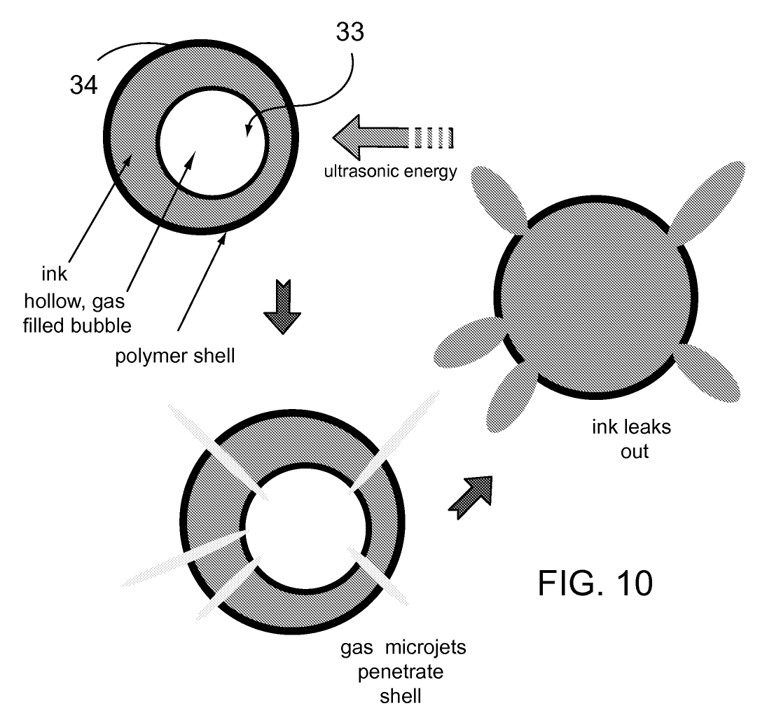

The ultrasonically active portion of the ink particle in the above examples is not restricted to porous sol-gel particles nor is the outer shell of the delivery vehicle restricted to a polymer. Furthermore, the ink can be freely suspended or contained in a porous matrix. For example, a nested bubble structure, with a gas bubble encapsulated within the polymer shell with the ink, ultrasound energy may be used to collapse the bubble, releasing microjets which penetrate the polymer shell as shown in FIG. 10. Alternatively, the bubble could be filled with a pH lowering gas such as CO2 to neutralize pH sensitive inks.

While inks were used as an example of the possible manipulations of ultrasonically active microparticles 10 for ease in conceptual understanding, a more serious example is a therapeutic application of a delivery structure 20 or 28, which includes delivery and application of a drug or other substance. An aqueous solution of a plurality of ultrasonically active microparticles 10 carrying a delivery structure (20, 28) containing a drug or other substance can be injected intravenously in a subject. Ultrasonic imagery can be used to trace the progress of microparticles 10 to a desired location, organ, injury, thrombosis, tumor and the like. Once sufficient amounts have accumulated as can be seen by the ultrasonic imagery, the drug can be released as described previously, using high levels of sound energy. Additionally, ultrasonically active microparticles 10 can be manipulated as previously described by using an externally applied ultrasound field. Manipulation of ultrasonically active microparticles 10 includes movement, release of substances and catastrophic destruction. All of which can be useful therapeutically.

Various changes and modifications to the embodiments herein chosen for purposes of illustration will readily occur to those skilled in the art. To the extent that such modifications and variations do not depart from the spirit of the invention, they are intended to be included within the scope thereof, which is assessed only by a fair interpretation of the following claims.

Having fully described the invention in such clear and concise terms as to enable those skilled in the art to understand and practice the same, the invention claimed is:

1. An ultrasonically active microparticle comprising:
a porous interior particle having hydrophobic pores;
a gas in the hydrophobic pores; and
a hydrophilic exterior shell surrounding the interior particle, forming an ultrasonically active microparticle and allowing the microparticle to be suspended in an aqueous solution.

2. An ultrasonically active microparticle as claimed in claim 1 having a size in the range of 0.1 to 20 microns.

3. An ultrasonically active microparticle as claimed in claim 1 further including hydrophobic materials incorporated into the pores to prevent filling of the pores in an aqueous solution.

4. An ultrasonically active microparticle as claimed in claim 1 incorporating a cleavable/hydrolysable linker to accelerate degradation of the matrix in aqueous fluids.

5. An ultrasonically active microparticle as claimed in claim 1 further comprising a delivery structure containing a substance, the delivery structure carried by the ultrasonically active microparticle.

6. An ultrasonically active microparticle as claimed in claim 5 wherein the delivery structure includes at least one nanopacket containing the substance, the nanopacket attached to the outer shell of the ultrasonically active microparticle.

7. An ultrasonically active microparticle as claimed in claim 5 wherein the delivery structure includes a delivery shell enclosing the microparticle and the substance.

8. An ultrasonically active microparticle as claimed in claim 7 wherein the delivery shell is a lipid membrane.

9. An ultrasonically active microparticle as claimed in claim 1 wherein the ultrasonically active microparticle is manipulable by using an externally applied ultrasound field.

10. A method of fabricating an ultrasonically active microparticle comprising the steps of:
forming a porous interior particle having hydrophobic pores;
inserting a gas into the hydrophobic pores; and
surrounding the interior particle with a hydrophilic exterior shell.

11. A method of fabricating an ultrasonically active microparticle wherein the step of forming a porous interior particle further comprises incorporating hydrophobic materials into the pores to prevent filling of the pores in an aqueous solution.

12. A method of fabricating an ultrasonically active microparticle wherein the step of forming a porous interior particle further comprises employing a sol-gel process to form an aerogel and reducing the aerogel to particles having a size in the range of 0.1 to 20 microns.

13. A method of using ultrasonically active microparticles comprising the steps of:
providing a plurality of ultrasonically active microparticle in an aqueous solution, each ultrasonically active microparticle comprising:
a porous interior particle having hydrophobic pores;
a gas in the hydrophobic pores; and
a hydrophilic exterior shell surrounding the interior particle, forming an ultrasonically active microparticle and allowing the microparticle to be suspended in the aqueous solution;
introducing the solution of ultrasonically active microparticles to a desired site.

14. A method of using ultrasonically active microparticles as claimed in claim 13 wherein the step of introducing includes injecting the solution intravenously.

15. A method of using ultrasonically active microparticles as claimed in claim 14 wherein the step of introducing further comprises ultrasonically imaging the desired site to determine the number of ultrasonically active microparticles in position.

16. A method of using ultrasonically active microparticles as claimed in claim 13 further comprising the step of manipulating the ultrasonically active microparticles by using an externally applied ultrasound field.

17. A method of using ultrasonically active microparticles as claimed in claim 13 wherein the step of providing a plurality of ultrasonically active microparticles in an aqueous solution further comprises providing at least some of each of the plurality of ultrasonically active microparticles with a delivery structure containing a substance, the delivery structure carried by the ultrasonically active microparticle.

18. A method of using ultrasonically active microparticles as claimed in claim 17 wherein the delivery structure includes at least one nanopacket containing the substance, the nanopacket attached to the outer shell of the ultrasonically active microparticle, the substance released by using an externally applied ultrasound field.

19. A method of using ultrasonically active microparticles as claimed in claim 18 wherein the nanopacket is selected from a group consisting of a liposome, a solid lipid nanoparticle, an antioxidant nanoparticle, and a hollow shell comprising polymers, saccharides, or combinations of polymers and lipids, surrounding the substance.

20. A method of using ultrasonically active microparticles as claimed in claim 17 wherein the delivery structure includes a delivery shell enclosing the microparticle and the substance, the substance released by using an externally applied ultrasound field.
